SEQUENCE LISTING (6 sequences)

SEQ ID NO: 1 - DNA, 7470 bp, Artificial Sequence
  pShuttle-CMV vector
gctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggatcgaattaattcttaattaacatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatactgtaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatctggtaccgtcgacgcggccgctcgagcctaagcttctagataagatatccgatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcggatctgggcgtggttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagagggagtttaaacgaattcaatagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttaattaacatgcatggatccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcagccatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattttgttaaaatttttgttaaatca

SEQ ID NO: 2 - DNA, 648 bp, Artificial Sequence
  RSB kozak-2019 nCoV-Flag/His
aaggaggccgccaccatgaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgttgactataaggatgacgacgataaaggacatcatcaccatcatcactga

SEQ ID NO: 3 - DNA, 8070 bp, Artificial Sequence
  pShuttle-CMV-RSB kozak-2019 nCoV-Flag/His
gctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggatcgaattaattcttaattaacatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatactgtaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatctggtaccgtcgacaaggaggccgccaccatgaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgttgactataaggatgacgacgataaaggacatcatcaccatcatcactgatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcggatctgggcgtggttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagagggagtttaaacgaattcaatagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttaattaacatgcatggatccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcagccatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattttgttaaaatttttgttaaatca

SEQ ID NO: 4 - DNA, 15 bp, Artificial Sequence
  RSB kozak
aaggaggccgccacc

SEQ ID NO: 5 - protein, 9 aa, Artificial Sequence
  RSB kozak-2019 nCoV-Flag/His - Flag
DYKDDDDKG

SEQ ID NO: 6 - protein, 6 aa, Artificial Sequence
  RSB kozak-2019 nCoV-Flag/His - His
HHHHHH

(19) United States
(12) Patent Application Publication
(10) Pub. No.: US 2022/0265814 A1
(43) Pub. Date: Aug. 25, 2022

(54) RECOMBINANT ADENOVIRUS VACCINE FOR CORONA VIRUS DISEASE 19 AND COMBINATION THERAPY USING THE SAME

(71) Applicant: GENEUIN-TECH CO., LTD., Gimhae-si, (KR)

(72) Inventor: Eun Yeong HAN, Seoul (KR)

(73) Assignee: GENEUIN-TECH CO., LTD., Gimhae-si (KR)

(21) Appl. No.: 17/408,489

(22) Filed: Aug. 23, 2021

(30) Foreign Application Priority Data
Feb. 24, 2021 (KR) 10-2021-0024628

(51) Int. Cl. A61K 39/215; C12N 15/86; C07K 14/005; A61K 31/19; A61P 31/14; A61P 37/04

(52) U.S. Cl. CPC A61K 39/215; C12N 15/86; C07K 14/005; A61K 31/19; A61P 31/14; A61P 37/04; C12N 2710/10043; C12N 2770/20022; C12N 2770/20034; C12N 2770/20071; A61K 2039/543

(57) ABSTRACT

The present invention relates to a live recombinant adenovirus vaccine and a combination therapy using the same for preventing infection of coronavirus infectious disease-19 (COVID-19), which occurred in Wuhan, China in 2019, and a rapid and harmless COVID-19 vaccine can be developed by producing an antibody specific to the novel coronavirus antigen using the recombinant adenovirus according to the present invention. In addition, through the combination therapy of the recombinant adenovirus vaccine and the compound according to the present invention, it is possible to more effectively and safely prevent or treat viral diseases such as COVID-19 as well as herpes simplex virus (HSV) infection.

### CROSS-REFERENCE TO RELATED APPLICATION

This application claims priority to and the benefit of Korean Patent Application No. 10-2021-0024628 filed in the Korean Intellectual Property Office on Feb. 24, 2021, the entire contents of which are incorporated herein by reference.

### BACKGROUND OF THE DISCLOSURE

### 1. Field of the Disclosure

The present invention relates to a live recombinant adenovirus vaccine and combination therapy using the same for preventing infection of coronavirus infectious disease-19 (COVID-19) that occurred in Wuhan, China in 2019.

### 2. Description of the Related Art

Coronavirus infectious disease-19 (COVID-19) is a respiratory infectious disease that first appeared in Wuhan, China in December 2019 and then spread to the world. The World Health Organization (WHO) announced on Jan. 9, 2020 that the cause of the pneumonia was a new type of coronavirus (SARS-CoV-2, named February 11 by the International Classification of Viruses), and the pathogen was identified. Coronavirus infectious disease-19 is known to be transmitted by the penetration of droplets (saliva drop) of an infected person into the respiratory tract or the mucous membranes of the eyes, nose, and mouth, and the main symptoms after an incubation period of about 2 to 14 days (estimated) are fever (37.5° C.), respiratory symptoms such as cough or shortness of breath, and pneumonia, and although rare, there are cases of asymptomatic infection.

However, little is known about the pathogenesis of this disease worldwide, and there is no vaccine or therapeutic agent. With the advent of a new type of virus that is difficult to predict, there is a shortage of preventive vaccines and the rapid production of viruses for vaccines is required.

Accordingly, the present inventors intend to make a live vaccine using a recombinant adenovirus that produces various types of viral antigens from a single viral particle. Recombinant adenovirus can easily enter the living body only by nasal inhalation. Therefore, antigen-specific antibodies can be easily and quickly obtained, and there is no risk of zoonotic infection, so it is safe to use in the human body. It is possible to quickly and safely develop an antibody based on a recombinant adenovirus vector when a new infectious disease like now is prevalent, thereby preventing a shortage of preventive vaccines and diagnostic kits. Early detection and early treatment of new infectious diseases can prevent secondary infection and accelerate the patient's return to society. In addition, it will be possible to minimize economic and industrial losses by preventing damages such as quarantine measures for two weeks of contacts and temporary closure of restaurants, branches, and companies by identifying the movement route of the infectious disease-infected person.

### PRIOR ART LITERATURE

### Patent Literature

Chinese patent publication No. 112206318 (published on Dec. 1, 2021)

### SUMMARY OF THE DISCLOSURE

It is an object of the present invention to provide a vaccine composition for preventing or treating coronavirus infectious disease-19 (COVID-19).

It is another object of the present invention to provide a pharmaceutical composition for preventing or treating COVID-19 through the combination therapy of the COVID-19 vaccine and the compound.

It is another object of the present invention to provide an antiviral pharmaceutical composition effective for treating adenovirus infection or herpes simplex virus (HSV) infection.

In order to achieve the above object, the present invention provides a vaccine composition for COVID-19 using a recombinant adenovirus. In addition, the present invention provides a pharmaceutical composition of combination therapy for the prevention or treatment of COVID-19 through the combination therapy of the vaccine composition and a specific compound.

In addition, the present invention provides an antiviral pharmaceutical composition effective for the treatment of adenovirus infection or herpes simplex virus (HSV) infection.

### BRIEF DESCRIPTION OF THE DRAWINGS

FIG. 1 shows the sequence identity with other similar coronavirus proteins of SARS-coronavirus-2 (SARS-CoV-2), the virus that causes COVID-19.

FIG. 2 shows the spike protein sequence of SARS-CoV-2.

FIG. 3 shows the spike protein sequence of SARS (Severe Acute Respiratory Syndrome) (in this case, the red highlighted portion indicates the predicted site of the binding domain of SARS-CoV-2).

FIG. 4 shows the sequence similarity of the spike protein between SARS-CoV-2 and SARS virus, confirming that 83% identical.

FIG. 5 shows the antigen gene information used in the present invention in which the RBS kozak sequence inserted at the 5′-terminus and the Flag/His (RBS kozak-2019 nCoV-Flag/His) sequence inserted at the 3′-end at sites 330-524 of the RBD are shown.

FIG. 6 shows recombinant protein expression according to an embodiment of the present invention, which indicates that in all four candidate groups (Lane 1-4) inserting the COVID-19 antigen into the recombinant adenovirus platform, the COVID-19 antigen having the size of about 25 kDa is normally expressed.

FIGS. 7A and 7B show the efficacy of the COVID-19 antibody against recombinant adenovirus according to an embodiment of the present invention; FIG. 7A shows a result of confirming the antigen inhibitory effect at 24, 48, 72 hours by treating with the antibody at 100 μM and 200 μM; and FIG. 7B shows a result of confirming the antigen action inhibitory effect at 24, 48, and 72 hours by treating with the antibody at 200 μM, 400 μM, and 600 μM.

FIG. 8 shows the formation of antibodies reacting with recombinant proteins in plasma of mice inhaled with recombinant adenovirus according to the present invention.

FIG. 9 shows the structure of the compound examining the antiviral activity in an embodiment of the present invention.

FIG. 10, FIG. 11 and FIG. 12 show the results of the antiviral effect after 24 hours, 48 hours, and 72 hours, respectively, after treatment with the compound according to Table 1.

FIG. 13 shows the antiviral effect of C2, C6, C8, and C10 on adenovirus after 24 hours, 48 hours and 72 hours.

FIG. 14 shows the antiviral effect of C2, C6, C8, and C10 on herpes simplex virus (HSV) after 24 hours, 48 hours and 72 hours.

FIG. 15A and FIG. 15B are graphs showing the antiviral effect on COVID-19 according to the combination treatment of the recombinant adenovirus vaccine and ECCAA according to the present invention.

### DETAILED DESCRIPTION OF THE EMBODIMENTS

Hereinafter, the present invention will be described in detail.

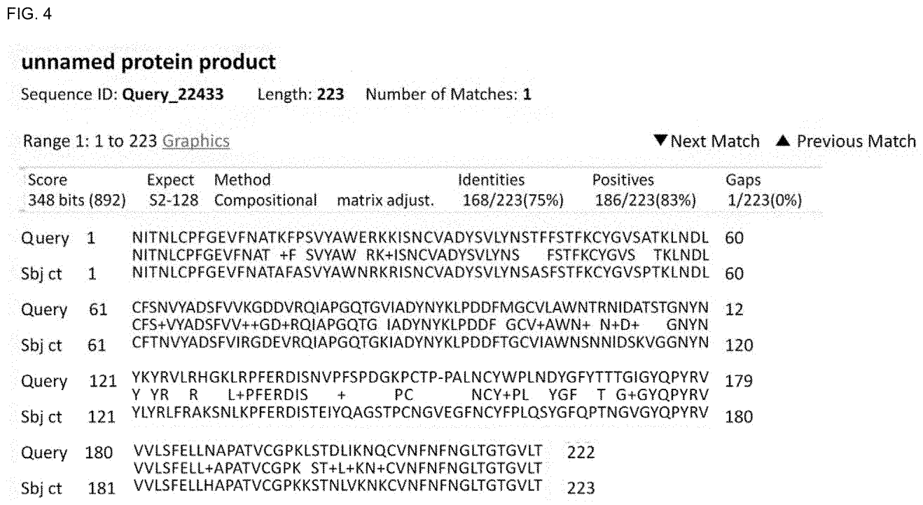

The present inventors have confirmed that 80% of the virus of coronavirus infectious disease-19 (COVID-19) are similar to Severe Acute Respiratory Syndrome (SARS) virus, and 83% of the spike protein related to the neutralizing antibody are the same as it, in FIG. 4, and have developed a safe live recombinant adenovirus vaccine by inserting the same into a recombinant adenovirus vector platform as an antigen, and have found that the therapeutic effect of COVID-19 can be enhanced by using the live vaccine with a novel antiviral agent in combination, and have completed the present invention.

The present invention provides a recombinant expression vector wherein gene sequence from sites 330 to 524 of receptor binding domain (RBD) of the spike protein (S protein) on a surface of SARS-CoV-2 and a ribosome binding site (RBS) sequence at 5′-end of the gene sequence and tag gene sequence at 3′-end of the gene sequence are inserted.

More preferably, the recombinant expression vector is prepared by inserting gene sequence from sites 330 to 524 of receptor binding domain (RBD) of the spike protein (S protein) on a surface of SARS-CoV-2 and a ribosome binding site (RBS) kozak sequence at 5′-end of the gene sequence and Flag/His as a tag gene at 3′-end of the gene sequence.

The ribosome binding site may be RBS/Kozak (aaggaggccgccacc (SEQ ID NO: 4)) that helps to detoxify the protein, and the tag gene serves to facilitate the purification of the target protein for expression, and can be a Flag tag (DYKDDDDKG (SEQ ID NO: 5)) and His tag (HHHHHH (SEQ ID NO: 6)) gene, however, it is not limited thereto.

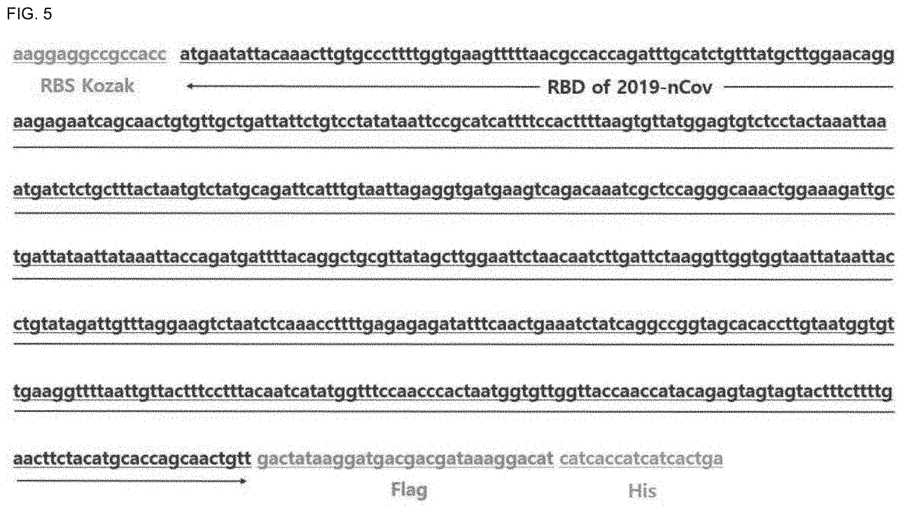

The antigen gene introduced into the recombinant expression vector according to the present invention is as shown in FIG. 5, and may be the nucleotide sequence of SEQ ID NO: 2.

As used herein, “vector” refers to a self-replicating DNA molecule used to carry a clonal gene (or another piece of clonal DNA).

As used herein, “expression vector” refers to a recombinant DNA molecule comprising a desired coding sequence and an appropriate nucleic acid sequence essential for expressing a coding sequence operably linked in a specific host organism. The expression vector may preferably comprise one or more selectable markers. The marker is a nucleic acid sequence having characteristics that can be selected by a conventional chemical method, and includes all genes that can distinguish transformed cells from non-transformed cells. Examples include antibiotic resistance genes such as ampicillin, kanamycin, geneticin (G418), bleomycin, hygromycin, chloramphenicol, but they are limited thereto and they can be appropriately selected by those skilled in the art. To express the DNA sequences of the present invention, any of a wide variety of expression control sequences can be used in the vector. Examples of useful expression control sequences include construction and other sequences derived therefrom and various combinations thereof, known to regulate the expression of genes of for example, early and late promoters of SV40 or adenovirus, promoters and enhancers of CMV, LTR of retrovirus, lac system, trp system, TAC or TRC system, T3 and T7 promoters, major operator and promoter regions of phage lambda, regulatory regions of fd coding proteins, promoters for 3-phosphoglycerate kinase or other glycolytic enzymes, promoters of said phosphatases, such as Pho5, promoters of yeast alpha-crossing system and prokaryotic or eukaryotic cells or viruses thereof.

In addition, the present invention provides a recombinant strain transformed with the recombinant expression vector. Preferably, the recombinant strain may be an adenovirus, but it is not limited thereto.

In addition, the present invention provides a SARS-coronavirus-2 (SARS-CoV-2) recombinant protein obtained from the recombinant strain.

As used herein, “recombinant protein” means that one or more polypeptides are bound to a single-stranded polypeptide through a peptide bond, and it refers to a recombinant protein produced by fusing at least one genes including a target gene by a recombinant method and translating into a single polypeptide. In the present invention, the recombinant protein has a target protein or peptide sequence fused to each other, and includes an amino acid cleavage sequence (chemicals or enzyme specific cleavage sequence) that can be specifically recognized and cleaved by a chemical or enzyme between the target protein or peptide sequences.

In addition, the present invention provides a method of preparing recombinant adenovirus particles comprising transfecting the adenovirus with the recombinant expression vector; and culturing the transfected adenovirus. Preferably, the recombinant adenovirus particle can express a SARS-CoV-2 (SARS-CoV-2) recombinant protein.

In addition, the present invention provides a vaccine composition for preventing or treating COVID-19 comprising a recombinant adenovirus obtained by transfecting and culturing the adenovirus with the recombinant expression vector.

Host animals to which the vaccine of the present invention can trigger an immune response may include humans, dogs, cats, pigs, horses, chickens, ducks, turkeys, ferrets and the like, but they are not limited thereto.

The vaccine of the present invention may be an attenuated live or dead vaccine, a subunit vaccine, a synthetic vaccine, or a genetic engineering vaccine, but a live vaccine that induces an effective immune response is preferable.

In the present invention, “live attenuated vaccine” or “live vaccine” means a vaccine comprising a live virus active ingredient. In addition, “attenuation” refers to artificially weakening the toxicity of a living pathogen, and inducing immunity by mutating genes involved in essential metabolism of pathogens, thereby not causing disease in the body, and stimulating only the immune system. Attenuation of the virus can be achieved by ultraviolet (UV) irradiation, chemical treatment, or high-order serial subculture in vitro. Attenuation can also be achieved by making distinct genetic changes, for example by specific deletions of viral sequences known to confer virulence or insertion of the sequences into the viral genome.

In addition, the vaccine of the present invention may further include one or more selected from the group consisting of a solvent, an adjuvant, and an excipient. The solvent includes physiological saline or distilled water, and the adjuvant includes Freund's incomplete or complete adjuvant, aluminum hydroxide gel and vegetable and mineral oils, and the excipient includes aluminum phosphate and aluminum hydroxide or aluminum potassium sulfate, but it is not limited thereto, and may further include substances used in the preparation of vaccines well known to those skilled in the art.

In addition, the vaccine of the present invention may be prepared in an oral or parenteral formulation, and may be administered by an intradermal, intramuscular, intraperitoneal, nasal or epidural route. Preferably, the vaccine of the present invention may be administered nasally or nasally inhaled, but it is not limited thereto.

In addition, the present invention provides a method of preventing or treating COVID-19 comprising administering the vaccine composition to a subject other than humans.

In the present invention, the term “subject” refers to any animal, including humans that has already been infected with or may be infected with an influenza virus. The disease can be effectively prevented and treated by administering the vaccine composition of the present invention to a subject. For example, the composition of the present invention can treat humans infected with various COVID-19 virus subtypes or variants of the COVID-19 virus. In addition, the composition of the present invention can treat chickens or pigs infected with various COVID-19 virus subtypes or variants of the COVID-19 virus. In the present invention, “prevention” means any action that suppresses or delays the onset of COVID-19 by the administration of a vaccine composition. In addition, “treatment” means any action that improves or beneficially changes symptoms caused by COVID-19 by the administration of a vaccine composition.

The vaccine composition of the present invention is administered in a pharmaceutically effective amount. The term “pharmaceutically effective amount” means an amount sufficient to treat a disease with a reasonable benefit/risk ratio applicable to medical treatment, and effective dosage levels can be determined according to factors including type and severity of the subject, age, sex, type of virus infected, drug activity, sensitivity to drug, administration time, route of administration, excretion rate, duration of treatment, concomitant drugs, and other factors well known in the medical field. The vaccine composition of the present invention may be administered as an individual therapeutic agent or may be administered in combination with other therapeutic agents, and may be administered sequentially or simultaneously with conventional therapeutic agents. In addition, it may be administered single or multiple. Taking all of the above factors into consideration, it is important to administer an amount that can obtain the maximum effect with a minimum amount without side effects, and can be easily determined by those skilled in the art.

In addition, the vaccine composition according to the present invention can be used in combination with the following compounds to further enhance the effect of treating or preventing COVID-19.

That is, the present invention provides a pharmaceutical composition for preventing or treating coronavirus infectious disease-19 (COVID-19) comprising: the vaccine composition; and at least one compound selected from the group consisting of ceanothic acid, 3-O-vanillyl ceanothic acid, epiceanothic acid 2-methyl ester and a compound represented by the following Chemical

Formula 1:

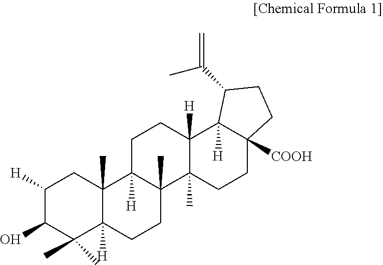

In addition, the present invention provides a pharmaceutical composition for preventing or treating Herpes simplex virus infection comprising: the vaccine composition; and at least one compound selected from the group consisting of ceanothic acid, 3-O-vanillyl ceanothic acid, epiceanothic acid 2-methyl ester and a compound represented by the above Chemical Formula 1.

In addition, the present invention provides an antiviral pharmaceutical composition comprising at least one compound selected from the group consisting of ceanothic acid, 3-O-vanillyl ceanothic acid, epiceanothic acid 2-methyl ester and a compound represented by the above Chemical Formula 1.

The compound may exhibit antiviral activity against adenovirus or Herpes simplex virus infection, but it is not limited thereto.

Hereinafter, the present invention will be described in more detail through examples. These examples are only intended to illustrate the present invention in more detail, and it will be apparent to those skilled in the art that the scope of the present invention is not limited by these examples according to the gist of the present invention. The examples of the present invention are provided to more completely explain the present invention to those of ordinary skill in the art.

### EXAMPLE 1

### Preparation of Live COVID-19 Recombinant Adenovirus Vaccine

The spike protein of COVID-19 with host-binding ability was obtained and it was inserted into the recombinant adenovirus, and the expression of the antigen in the cell was confirmed through an in vitro experiment.

1. Preparation of Antigen Gene

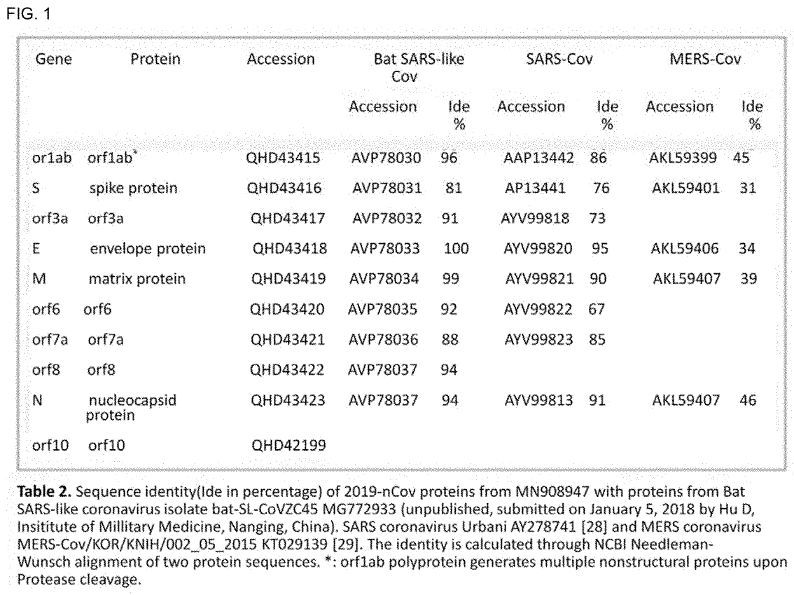

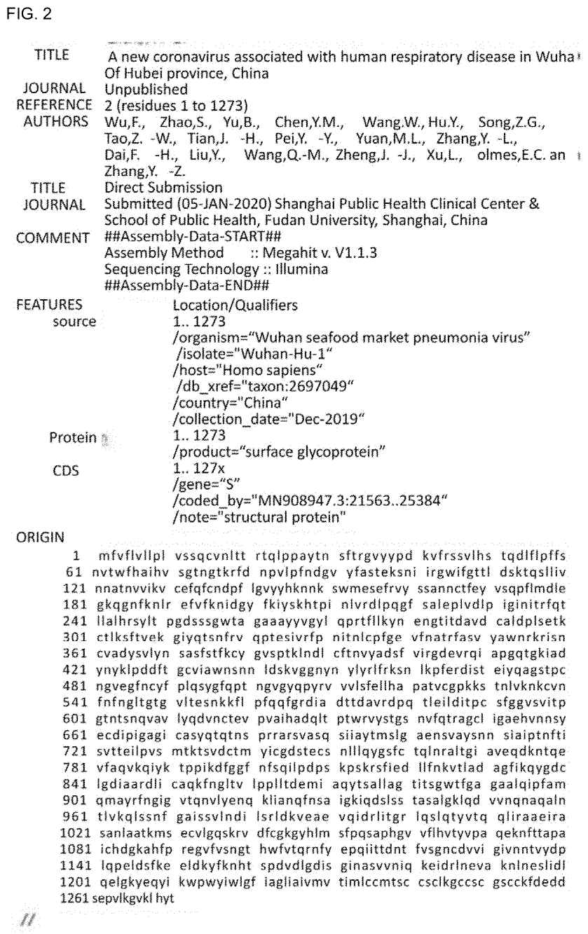

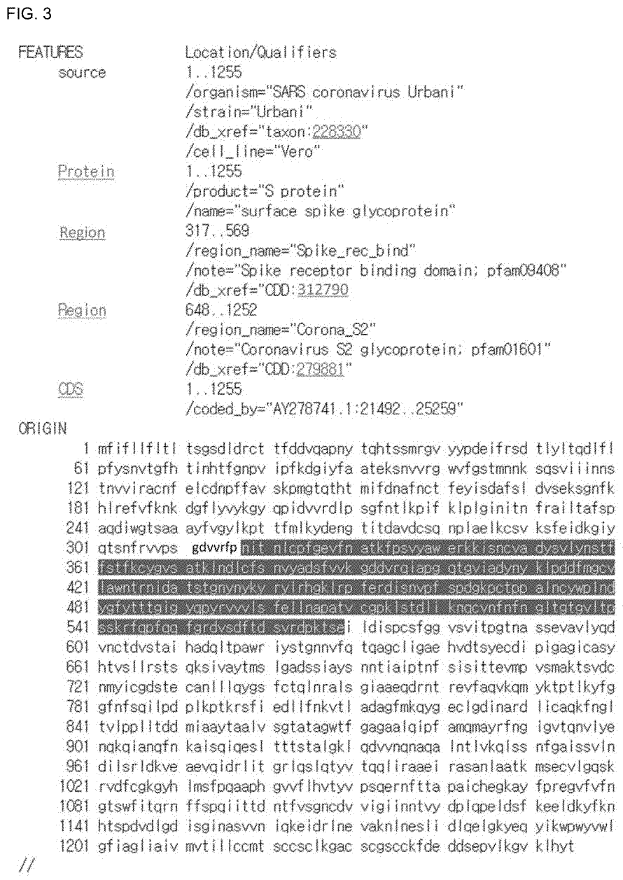

FIG. 1 confirms the sequence identity of COVID-19 with other similar coronavirus proteins according to an embodiment of the present invention, FIG. 2 shows the spike protein sequence of COVID-19 (ACCESION: QHD43416.1). In the present invention, sites 330-524 of the receptor binding domain (RBD) in the spike protein sequence were used as antigens. FIG. 3 shows the spike protein sequence (AAP13441.1) of severe acute respiratory syndrome (SARS). FIG. 4 confirms the sequence similarity of the spike protein between SARS-CoV-2 and SARS virus, it was confirmed that 83% is identical to each other.

The DNA sequence of pShuttle-CMV which is a recombinant adenovirus platform according to an embodiment of the present invention, and it is represented by SEQ ID NO: 1.

The antigen gene used in the present invention is prepared by inserting RBS kozak sequence at the 5′-end and Flag/His (RBS kozak-2019 nCoV-Flag/His) at the 3′-end in sites 330-524 of RBD as shown in FIG. 5. More specifically, a protein composed of a total of 2,690 amino acids was designed by binding the peptide that functions as Flag tag (DYKDDDDKG (SEQ ID NO: 5)) and His tag (HHHHHH (SEQ ID NO: 6)) for use in the identification and separation process of proteins expressed on the carboxy terminals of the peptide amino acids linked in this way so that they can be effectively translated in eukaryotic cells or E. coli at the sites 330-524 of RBD.

In order to prepare a recombinant virus expressing the protein produced as the above in animal cells, the codon base sequence corresponding to the amino acid sequence of the protein was determined by optimizing the codon in the form most readable in animal cells. Finally, an antigen gene expressing a recombinant protein was synthesized by adding the RBS/Kozak nucleotide sequence which is a ribosome binding site that helps in protein translation, in front of the entire nucleotide sequence (SEQ ID NO: 2). The genes was synthesized using the gene synthesis service of Cosmogenetech Inc.

The thus prepared RBS kozak-2019 nCoV-Flag/His was inserted into pShuttle-CMV using SalI and XbaI restriction enzymes (SEQ ID NO: 3).

2. Confirmation of Recombinant Protein Expression Using Recombinant Adenovirus

In order to confirm the production of the recombinant protein from the antigen gene (RBS kozak-2019 nCoV-Flag/His) prepared as described above, the synthesized gene is inserted into a eukaryotic expression vector, and a recombinant adenovirus particles containing the expression vector were produced. The prepared fusion protein gene was first obtained as a pAd5-AI#4212 clone inserted between the CMV promoter of the pShuttle-CMV shuttle vector and the SV40 polyA site required for recombinant adenovirus production in the direction for gene transcription.

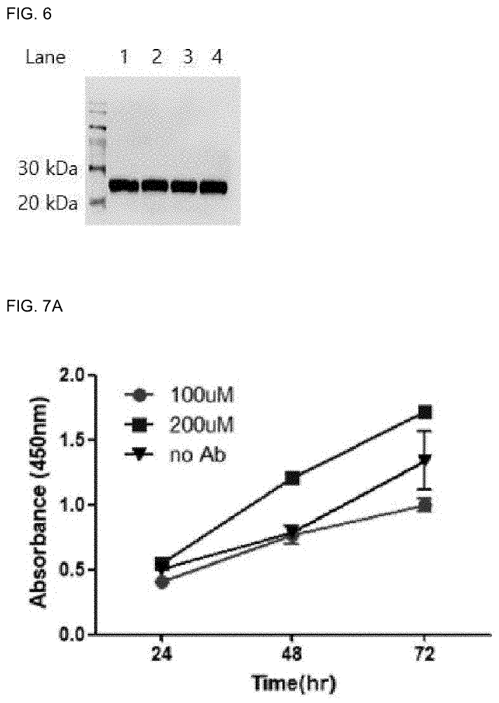

By requesting Geneuintech Co., Ltd., recombinant adenovirus particles containing the pAd5-AI#4212 DNA plasmid were prepared to investigate whether the fusion protein was produced in animal cells. To this end, 2×10⁵ HEK293A cells were cultured overnight, and then 2×10⁹ pfu of pAd5-AI#4212 recombinant adenovirus particles were mixed in the culture medium to obtain an MOU of 10,000, and the cells were infected and then incubated in a medium containing fetal bovine serum for 24 hours. Thereafter, the medium was exchanged with a medium without fetal bovine serum, and the culture solution was collected after culturing for 24 hours. In this way, the culture medium of the infected cells was collected a total of three times to recover the fusion protein. After the culture solution containing the collected recombinant protein was concentrated 20 times, the production of the recombinant protein was investigated by western blotting using a Flag tag. As a control, a recombinant adenovirus expressing green fluorescent protein (GFP) was used instead of the recombinant protein. After separating the concentrated culture solution by SDS-PAGE, the protein was transferred to NC membrane, reacted with a mouse anti-Flag antibody, and then reacted with a secondary antibody, Rat-anti-mouse IgG1-HRP (eBioscience). After the reaction was completed, it was washed with PBS, reacted with WEST-Queen™ kit (iNtRON), and the presence and size of the protein reacting with the Flag tag antibody was measured using a fluorescence image analyzer (Fusion-SL4) (FIG. 6).

As a result, it was confirmed that the COVID-19 antigen of about 25 kDa size was normally expressed in all four candidate groups (Lane 1-4) in which the COVID-19 antigen was inserted into the recombinant adenovirus platform according to the present invention as shown in FIG. 6.

3. Confirmation of Antibody Efficacy by Antigen-Antibody Polymerization

In the present invention, the efficacy of the commercial COVID-19 antibody against the COVID-19 recombinant adenovirus prepared in the Examples was confirmed.

That is, 1×10⁴ HEK293A cells were aliquoted and cultured in a 96-well plate, and a day later, they were infected with the COVID-19 recombinant adenovirus (MOI=0.5) prepared in Example. And one day later, each of the antibodies was treated by concentration, and after 1-3 days, Cyto X was treated, and the cell viability was measured by absorbance. The antibody used at this time was purchased and used [Cat: 40592-V05H, SARS-CoV-2 (2019-nCoV) Spike RBD-mFc Recombinant Protein (HPLC-verified)].

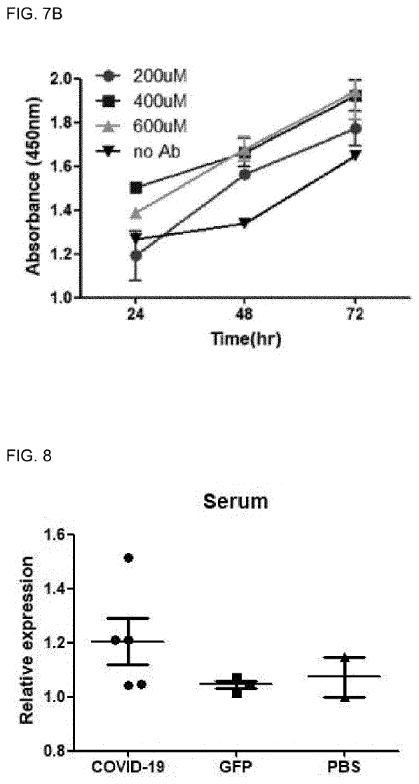

As a result, FIG. 7A shows that the antibody (Cat: 40592-V05H) at 100 μM and 200 μM was treated to confirm the effect of inhibiting antigen action at 24, 48, and 72 hours, and FIG. 7B shows the antibody at 200 μM, 400 μM, 600 μM was treated to confirm the effect of inhibiting antigen action at 24, 48, 72 hours.

4. Confirmation of Antibody Production by Recombinant Protein of COVID-19 Recombinant Adenovirus in Mice

In order to confirm the efficacy of the commercial COVID-19 antibody against the COVID-19 recombinant adenovirus according to an Example of the present invention, the formation of the COVID-19 antibody in the plasma of the mouse according to the recombinant adenovirus was confirmed. 5×10⁸ pfu recombinant adenovirus (COVID-19 antigen), 5×10⁸ pfu GFP and PBS were each injected by nasal inhalation. After 3 weeks, the same amount was administered in the same manner. After one week, blood was collected from the experimental animals, plasma was separated, and antibody formation was confirmed by ELISA.

As a result, it was confirmed that the antibody reacting with the recombinant protein was formed in the plasma of mice inhaling the recombinant adenovirus as shown in FIG. 8.

### EXAMPLE 2

### Review of Antiviral Effect Enhancement According to Combination Treatment of Live Recombinant Adenovirus Vaccine and Compound

1. Selection of Compounds Having Antiviral Activity Against Recombinant Adenovirus and Recombinant HSV

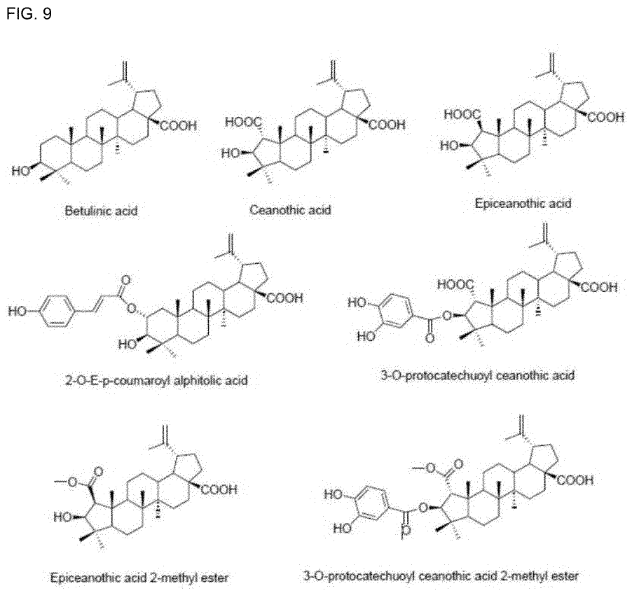

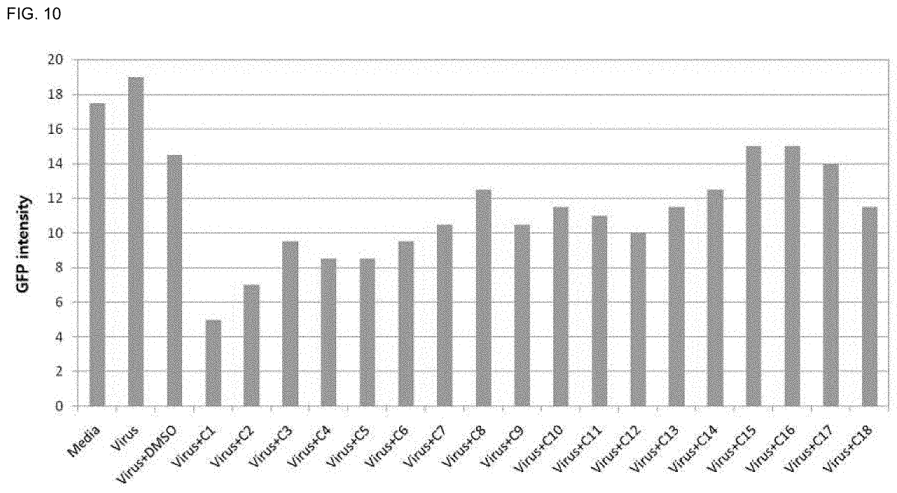

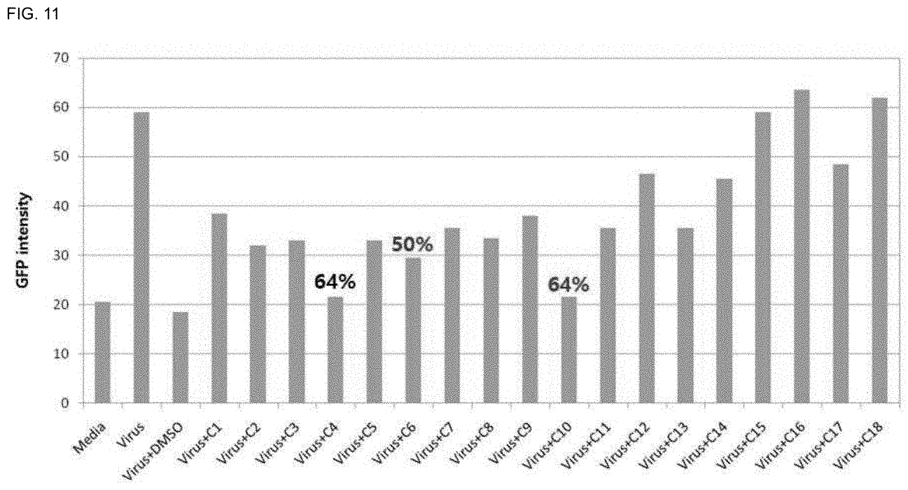

After dispensing HEK293A cells at 10,000 cells/well on a 96-well plate and stabilizing for 24 hours, the recombinant adenovirus (GFP tagging, MOI=0.5) prepared in Example and 18 compounds listed in Table 1 below (50 μM)) were treated and incubated for 24, 48 and 72 hours, the GFP fluorescence intensity (Excitation=485 nm, Emission=528 nm) was observed. In this case, the structure of the compound used was shown in FIG. 9.

TABLE 1

| Number | Compound Name |
|---|---|
| C1 | Betulinic acid |
| C2 | Ceanothic acid |
| C3 | Epiceanothic acid |
| C4 | 2-O-E-p-coumaroyl alphitolic acid |
| C5 | 3-O-protocatechuoyl ceanothic acid |
| C6 | 3-O-vanillyl ceanothic acid |
| C7 | 2-O-vanillyl alphitolic acid |
| C8 | epiceanothic acid 2-methyl ester |
| C9 | 3-O-protocatechuoyl ceanothic acid |
| | 2-methyl ester |
| C10 | ECCA A |
| C11 | ECCA B |
| C12 | vanillic acid |
| C13 | 6'-vanilloylisotachioside |
| C14 | Adouetine X |
| C15 | Jubanine F |
| C16 | Jubanine G |
| C17 | Jubanine H |
| C18 | Nummularine B |

In this case, ECCA A is a compound having the following structures:

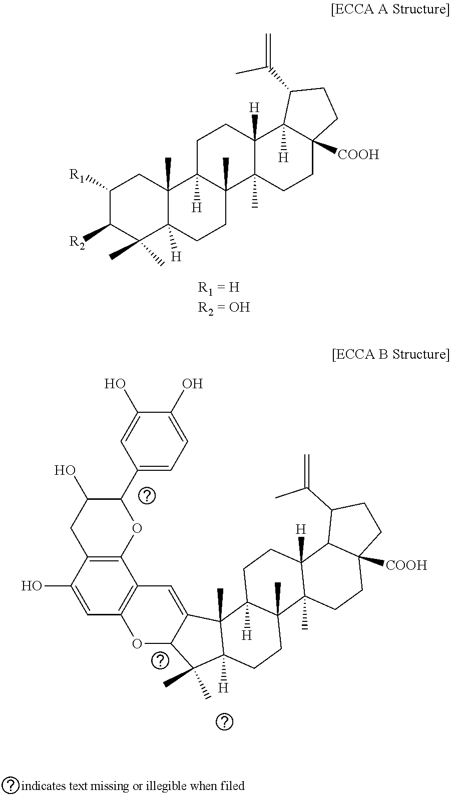

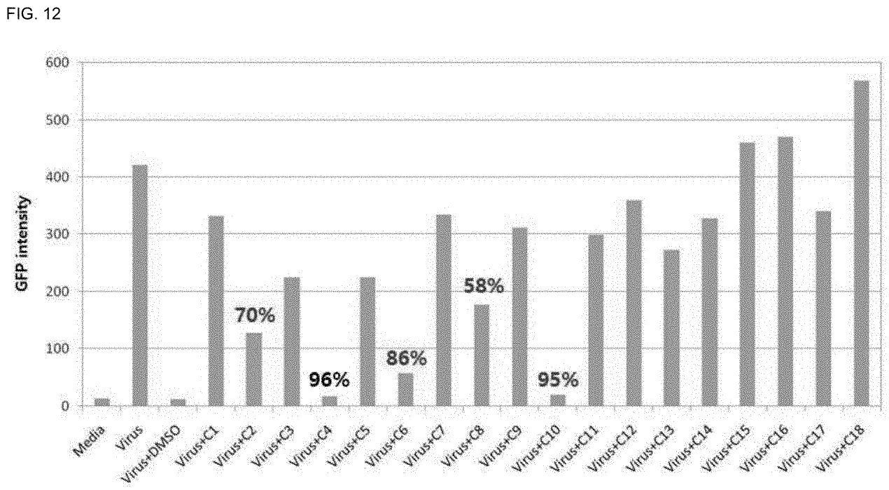

FIG. 10, FIG. 11 and FIG. 12 show the results of examining the antiviral effect after 24 hours, 48 hours and 72 hours, respectively, after treatment with the compounds of Table 1. In particular, when HEK293A cells were treated with C2, C6, C8, and C10, they showed excellent virus inhibitory activity of 50% or more after 72 hours, and C4 also exhibited excellent virus inhibitory activity, but 100% of cell death was confirmed after 72 hours of treatment and cytotoxicity was confirmed.

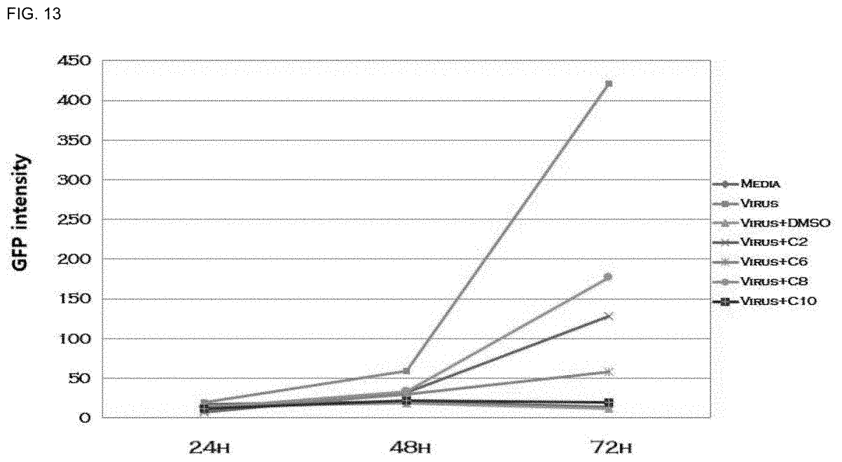

FIG. 13 is a summary of the antiviral effects of C2, C6, C8, and C10 after 24 hours, 48 hours and 72 hours, and it was confirmed that the antiviral activity of C10 was the most excellent.

2. Selection of Compounds with Antiviral Activity Against Herpes Simplex Virus (HSV)

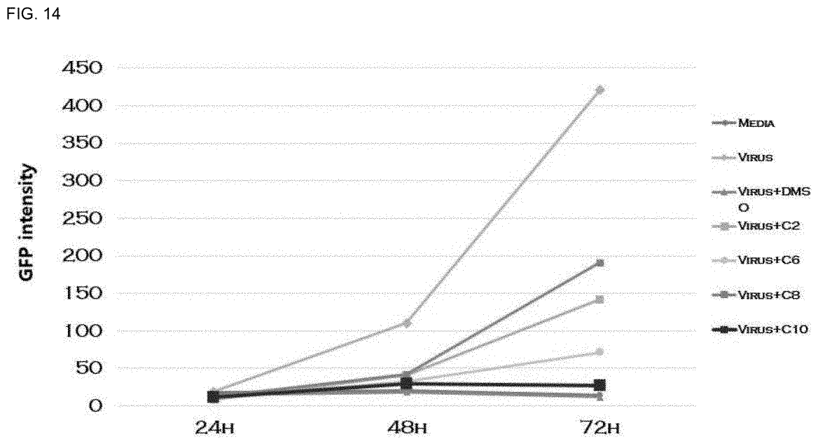

The experiment was performed in the same manner as in the previous experiment, except that the virus type was changed to HSV and the antiviral activity was examined. As a result, it was confirmed that the antiviral activity of C10 was the most excellent as shown in FIG. 14.

3. Confirmation of COVID19 Antibody-Antigen Polymerization Reaction According to Combination Treatment with ECCA A

First, 1×10³ HEK293A cells were aliquoted and cultured in a 96-well plate, and one day later, they were infected with 10⁶/ml of COVID-19 virus (COVID19 recombinant adenovirus). And one day later, 200 μM of the recombinant adenovirus according to an example of the present invention and 50 μM of ECCA A were treated, and 1-3 days later, Cyto X was treated and cell viability was measured by absorbance.

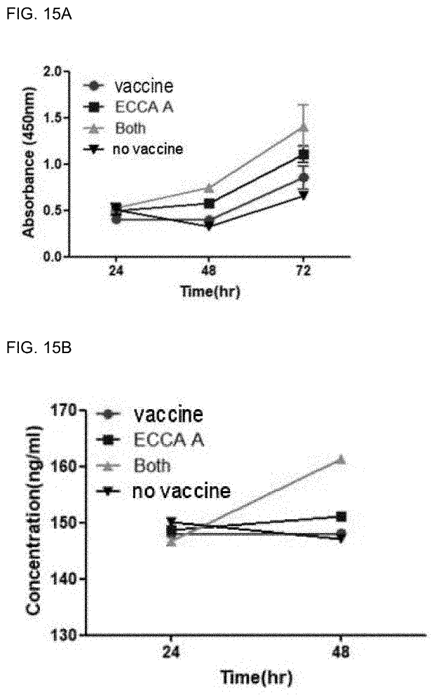

As a result, FIG. 15A is a graph showing the effect of the combination treatment of the recombinant adenovirus vaccine and ECCA A according to the present invention. An effective antigen-antibody polymerization reaction was confirmed by combination treatment with ECCA A, a novel antiviral compound. 15B is a graph showing the ability to bind to a fusion protein using a recombinant adenovirus by co-treating the recombinant adenovirus vaccine and ECCA A according to the present invention. It was confirmed that the binding to the antigen fusion protein was excellent when treated in combination with ECCA A.

According to the present invention, it is possible to rapidly and safely develop an antibody for COVID-19 based on a recombinant adenovirus vector that produces various types of viral antigens from one recombinant adenovirus particle and can easily enter the body only by nasal inhalation. It can prevent a shortage of preventive vaccines and diagnostic kits, easily diagnose the infectious disease, and prevent or treat COVID-19 more effectively and safely through combination therapy of compounds with antiviral activity.

By using the live recombinant adenovirus vaccine according to the present invention, secondary infection can be prevented through early detection and early treatment of COVID-19, and the patient's return to society can be accelerated, and It is possible to minimize economic and industrial losses by preventing damages such as quarantine measures for two weeks of contacts and temporary closure of restaurants, branches, and companies.

In addition, by using the recombinant adenovirus, it is possible to develop an antibody against a specific protein antigen as a practical platform that can properly respond to novel infectious diseases in addition to COVID-19.

While the present invention has been particularly described with reference to specific embodiments and drawings thereof, it is apparent that this specific description is only a preferred embodiment and that the scope of the present invention is not limited thereby to those skilled in the art. That is, the practical scope of the present invention is defined by the appended claims and their equivalents.

What is claimed is:

1. A recombinant expression vector wherein gene sequence from sites 330 to 524 of receptor binding domain (RBD) of spike protein (S protein) on a surface of SARS-coronavirus-2 (SARS-CoV-2) and a ribosome binding site (RBS) sequence at 5′-end of the gene sequence and tag gene sequence at 3′-end of the gene sequence are inserted.

2. The recombinant expression vector of claim 1, wherein antigen gene introduced into the recombinant expression vector comprises a DNA sequence of SEQ ID NO:2.

3. A recombinant strain transformed with the recombinant expression vector of claim 1.

4. The recombinant strain of claim 3, wherein the recombinant strain is an adenovirus.

5. SARS-coronavirus-2 (SARS-CoV-2) recombinant protein obtained from the recombinant strain of claim 4.

6. A vaccine composition for preventing or treating coronavirus infectious disease-19 (COVID-19) comprising a recombinant adenovirus obtained by transfecting and culturing adenovirus with the recombinant expression vector of claim 1.

7. The vaccine composition for preventing or treating coronavirus infectious disease-19 (COVID-19) of claim 6, wherein the recombinant adenovirus expresses SARS-coronavirus-2 (SARS-CoV-2) recombinant protein.

8. The vaccine composition for preventing or treating coronavirus infectious disease-19 (COVID-19) of claim 6, wherein the composition is nasally administered or nasally inhaled.

9. A pharmaceutical composition for preventing or treating coronavirus infectious disease-19 (COVID-19) comprising:
vaccine composition of claim 6; and
at least one compound selected from the group consisting of ceanothic acid, 3-O-vanillyl ceanothic acid, epiceanothic acid 2-methyl ester and a compound represented by Chemical Formula 1,

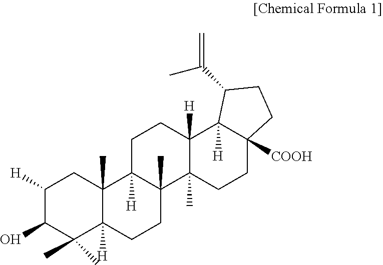

10. A pharmaceutical composition for preventing or treating Herpes simplex virus infection comprising:
vaccine composition of claim 6; and
at least one compound selected from the group consisting of ceanothic acid, 3-O-vanillyl ceanothic acid, epiceanothic acid 2-methyl ester and a compound represented by Chemical Formula 1,

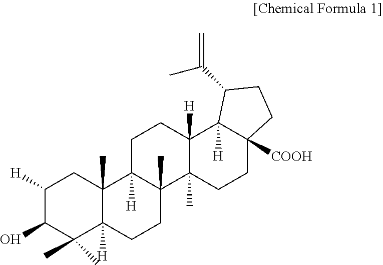

11. An antiviral pharmaceutical composition comprising at least one compound selected from the group consisting of ceanothic acid, 3-O-vanillyl ceanothic acid, epiceanothic acid 2-methyl ester and a compound represented by Chemical Formula 1,

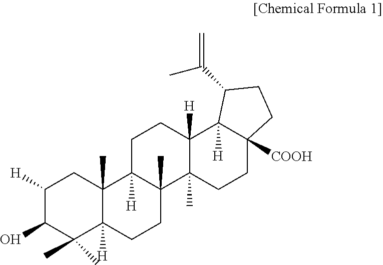

12. The antiviral pharmaceutical composition of claim 11, wherein the compound has antiviral activity against adenovirus or Herpes simplex virus infection.
